(19) United States
(12) Patent Application Publication
(10) Pub. No.: US 2011/0201063 A1
(43) Pub. Date: Aug. 18, 2011

(54) Algae growth for biofuels

(76) Inventor: Nickolaos Mitropoulos, Thomastown (AU)

(21) Appl. No.: 12/653,468

(22) Filed: Dec. 14, 2009

(30) Foreign Application Priority Data
Jun. 16, 2008 (AU) PCT/AU2008/000845
Jun. 14, 2009 (AU) 2007903190

(51) Int. Cl. C12P 7/64; C12N 1/12; C12M 1/00

(52) U.S. Cl. 435/134; 435/257.1; 435/292.1

(57) ABSTRACT

A method and apparatus of algal oil production including the steps of: control growing to provide intensive growth to supply starting means for algae farming; Farming algae from using primarily sunlight Processing algae from the farmed algae preferably using a wet extraction process: and wherein at least one of the steps includes use of a bag able to be interconnected to gas or liquid flows of at least one of water, CO₂, oxygen or air.

This application claims priority based upon PCT Application Ser. No. PCT/AU2008/000845 filed on Jun. 16, 2008, which claims priority on Australian application Ser. No. AU 2007903190, filed on Jun. 14, 2007, 2009.

### FIELD OF THE INVENTION

This invention relates to a method and system of algae growth for making bio fuels and particularly relates to an apparatus for enhancing algae growth for making bio fuels.

### BACKGROUND ART

Algae is a good source of biofuel because it grows rapidly, is rich in vegetable oil and can be cultivated in containers or ponds, minimising the use of land and fresh water.

Algae is a sustainable feedstock for production of diesel-type fuels with a very small CO₂ footprint.

Bio-diesel (alkyl esters) is a cleaner-burning diesel fuel made from natural, renewable sources such as virgin or recovered waste vegetable oils and can be directly substituted for diesel either as neat fuel (B100) or as an oxygenated additive (typically 5%-20%/B5 & B20). The largest producer and user of bio-diesel is Europe. It is usually made from rapeseed (canola) oil. Additional sources of feed-stocks for bio-diesel production include palm-oil, tallow and all waste lipids. In the United States, the second largest producer and user of bio-diesel, the fuel is usually made from soybean and corn oil.

However the use of food sources for biofuels is presently considered to be adding to the problem of the world food shortage.

Bio-diesel is registered as a fuel and fuel additive with the Environmental Protection Agency (EPA) in the USA. Bio-diesel is recognised by Federal and State governments as a valid alternative fuel.

The use of bio-diesel in a conventional diesel engine results in substantial reduction of unburned hydrocarbons, carbon monoxide, and particulate matter. The use of bio-diesel decreases the solid carbon fraction of particulate matter (since the oxygen in bio-diesel enables more complete combustion to CO₂), eliminates the sulphate fraction (as there is no sulphur in the fuel), while the soluble, or hydrocarbon, fraction stays substantially the same. Therefore, bio-diesel works well with new technologies such as catalysts (which reduces the soluble fraction of diesel particulate), particulate traps, and exhaust gas recirculation (giving potentially longer engine life due to less carbon).

While its emissions profile is lower, bio-diesel functions in the engine the same as petroleum diesel. Bio-diesel delivers emissions reductions while maintaining current fleets, refuelling stations, spare parts inventories and skilled diesel mechanics. Bio-diesel can be substituted for diesel with essentially no engine modifications, and maintains the payload capacity and range of diesel.

The use of bio-diesel is carbon-neutral. This can have significant financial benefits to users of bio-diesel as the “carbon trading” system begins to take effect.

Bio-diesel is safer for people to breathe. Research conducted in the United States showed bio-diesel emissions have significantly decreased levels of all target polycyclic aromatic hydrocarbons (PAH) and nitrated PAH compounds, as compared to petroleum diesel exhaust. PAH and nPAH compounds have been identified as potential cancer causing compounds. Results of the sub chronic inhalation testing showed no toxic results from bio-diesel exhaust emissions-even at the highest concentrations physically possible to achieve. These results conclusively demonstrate bio-diesel's health and environmental benefits as a non-toxic, renewable fuel.

Global tests sponsored by various governments and NGO's confirm that bio-diesel is less toxic than petroleum diesel and biodegrades as fast as dextrose (a test sugar). In addition, bio-diesel has a flash point of over 125° C. which makes it safer to store and handle than petroleum diesel fuel.

Depending on the application, climate and season the blend of bio-diesel can be from 2% up to 100%. In Europe (especially France), where low sulphur diesel has been in-place for many years, bio-diesel is added to provide the lubrication that was lost with the removal of the sulphur. In environmentally sensitive areas (marine, alpine) and in mines where the maximum environmental benefit is required, 100% bio-diesel is often used. In the US, where bio-diesel is in use in bus fleets, 20% bio-diesel is mostly used-to address the best current balance of emissions, cost and availability.

There are two common methods to grow algae.

The first uses a series of storage tanks linked by transparent tubes that rest on support structures. Algae and water are pumped through the pipes to ensure maximum exposure to sunlight. CO₂ piped into the installation feeds the algae. There is little risk of contamination of the algae as they are grown in a closed environment resembling laboratory conditions. Productivity per hectare is also high so the equipment takes up less land than open systems. However, the equipment is expensive since kilometres of tubes are necessary to produce commercial amounts of oil and maintenance costs are high to keep it clean and working.

The second uses a method of pumping water around a continuous loop of a man-made, open-air channel to expose the algae to sunlight. The raceways at existing open pond algae farms hold about as much water as a municipal swimming pool. Such open ponds are cheaper than closed systems, but they have their drawbacks too: light only reaches the algae near the surface, water easily evaporates and the temperature is harder to control. The risk of contamination is also greater than in closed systems. organisms that eat algae can enter open ponds.

It is therefore an object of the invention to provide a new apparatus and system that improves the growth and conversion of algal growth for obtaining natural oils to be used as biofuels.

It is also an object of the invention to provide a system that uses excess CO₂ and thereby improves carbon footprint of industry.

### SUMMARY OF THE INVENTION

In accordance with the invention there is provided a phyto bag for enhancing growth of algae for biofuels including:
a bag made from a substantially flexible sheet material enabling ready construction of large quantities;
the bag including a clear transparent top film to allow the passage of light to the algae in the bag; and
a metallic reflective bottom film to reflect light back to the algae in the bag
   wherein the bag has a large footprint relative to its height and the clear transparent top film and the metallic reflective bottom film improve sunlight and heat transfer to an algae material in the bag to enhance growth.

The invention also provides a system including:

a phyto bag having a large footprint relative to its height and including a top substantially translucent surface material

a sunlight controlling means on or above the top surface of the phyto bag; and

a heat aiding means on or below the bottom surface of the phyto bag; wherein the control of the sunlight controlling means and the heat aiding means ensures heat control within the phyto bag to ensure substantially heat in a predefined range.

In accordance with a form of the invention there is provided a sealed bag constructed with transparent metallic or reflective films for explicit purpose of growing algae resulting in harvesting of desired algal lipids and proteins.

The phyto bag in one embodiment of the invention includes:
a. a clear transparent top film to allow the passage of light to the algae in the bag
b. a metallic and reflective bottom film to reflect light back to the algae in the bag
c. multiple points of attachment to access contents which can be the form of liquids or gases
d. and when the top and bottom films have a medium to high oxygen barrier to capture oxygen produced by the algae
e. multiple fluid delivery means consisting of pipes and chambers positioned within the bag to maximise agitation
f. and minimum area of 1 sq m per phyto bag
g. a temperature maintenance system
h. a capacity to be linked with other bags via pumps and tanks to form a modular system

The bags will create a sealed modular network that will provide a controlled space to grow the algae of choice and maximise lipid and proteins production.

The phyto bags can be constructed with materials that are weatherproof and resist deterioration when exposed to the elements.

The phyto bag modular system consists of the plurality of bags that are interconnectable and in addition comprise:
i) aboveground tank fitted with heating and cooling options located on an elevated position to achieve maximum static head
ii) transfer pumps to effect fluid movements
iii) bags placed on flat and sloping ground over pipe heat exchangers
iv) below ground tank which would be the receival or harvesting tank.

The phyto bag modular system is duplicated according to the number of days for algae to grow to optimal concentration for the harvesting from the resulting modular system.

### BRIEF DESCRIPTION OF THE DRAWINGS

In order that the invention is more easily understood embodiments will be described by way of illustration with reference to the drawings wherein:

FIG. 1 is a diagrammatic view of an algal farming system in accordance with the invention;

FIG. 2 is a cross-sectional elevation of a form of phyto bag in accordance with an embodiment of the invention for use in the algal farming system of FIG. 1 and having integral internal air circulation;

FIG. 3 is a cross-sectional elevation of a second form of phyto bag in accordance with an embodiment of the invention for use in the algal farming system of FIG. 1 and having external air circulation;

FIG. 4 is a general diagrammatic view of a phyto bag in an algae farming system in accordance with an embodiment of the invention;

FIG. 5 is a general diagrammatic view of the phyto bag in an algae farming system in accordance with a still further embodiment of the invention;

FIG. 6 is a general diagrammatic view of the algae farming system in accordance with a further embodiment of the invention having three layers including solar bag, phyto bag and temperature control bag;

FIG. 7 is an overhead view of a temperature bag of FIG. 6 with numerous connecting inlets and outlets for connection to inlet and outlet feeds of gases and liquids;

FIG. 8 is an overhead view of a phyto bag of FIG. 6 with numerous connecting inlets and outlets for connection to inlet and outlet feeds of gases and liquids;

FIG. 9 is an overhead view of a solar bag of FIG. 6 with numerous connecting inlets and outlets for connection to inlet and outlet feeds of gases and liquids;

FIGS. 10 and 11 are perspective views of a modular control cell structure that can house a plurality of control bags for instigating initial algae growth for deployment in phyto bags in algae farming system of FIGS. 1 to 9;

FIG. 12 is an elevation of a control bag for use in the modular control cell structure of FIGS. 10 and 11 showing serpentine flow path;

FIG. 13 is an elevation of a cultivation bag for use in the modular control cell structure of FIGS. 10 and 11;

FIG. 14 is a flowchart of creation of biodiesel from algae;

FIG. 15 is a diagrammatic view of a modular system of algae farming in accordance with an embodiment of the invention and including powering from hybrid renewable energy system;

FIG. 16 is a general diagrammatic view of use of a settling tank in an algae farming system in accordance with the invention of FIG. 15;

FIG. 17 is a general diagrammatic view of use of a flocculation tank in an algae farming system in accordance with the invention of FIG. 15;

FIG. 18 is a plan view of a drying bag in an algae farming system in accordance with the invention;

FIG. 19 is a general diagrammatic view of use of a drying bag in an algae farming system in accordance with the invention;

FIG. 20 is a diagrammatic operational view of a drying bag in accordance with the invention; and

FIG. 21 is a flowchart of processing of algal material from the farm using the drying bag of FIGS. 18, 19, 20 in accordance with a full fat extraction process of the invention in which oil is left in the product;

FIG. 22 is a diagrammatic view of a temperature tank for use in the algae farming system in accordance with the invention;

FIG. 23 is a flowchart of processing of algal farming in accordance with the invention including a first wet extraction process;

FIG. 24 is a flowchart of processing of algal farming in accordance with the invention including a first wet extraction process;

### DESCRIPTION OF PREFERRED EMBODIMENTS OF THE INVENTION

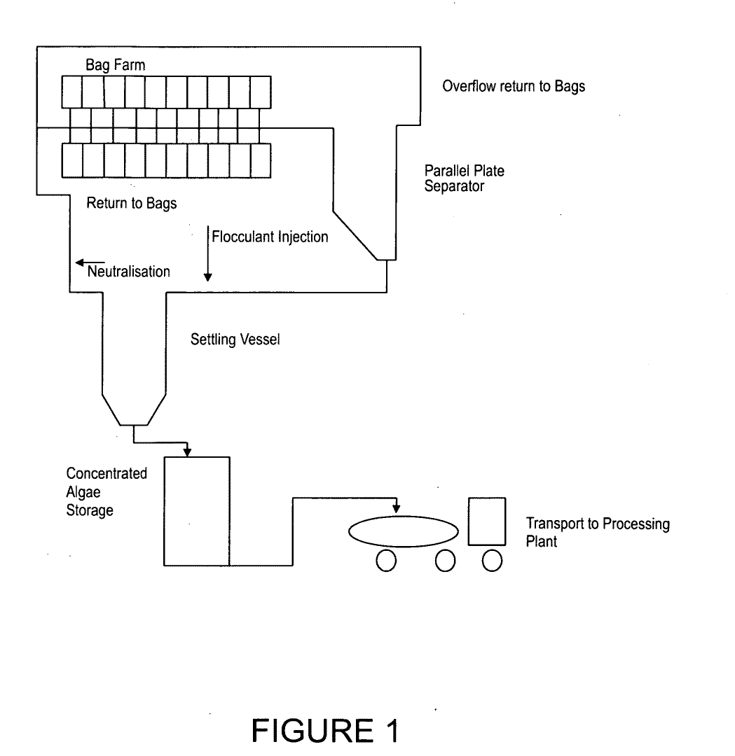

Referring to the drawings and particularly FIG. 1 there is shown an algae farming system that is modular and presently consists of 10 or more branches. Each of branches typically will have phyto bags. The growth of algae is controlled in batches so that in case of contamination any bag or branch can be isolated. The algal farming system is needed to be based on the use of low-cost methods certain to ensure that the upper economic reality of algae farming for the creation of biofuels is competitively comparable to the costs of oil based fuels from mined oil reserves.

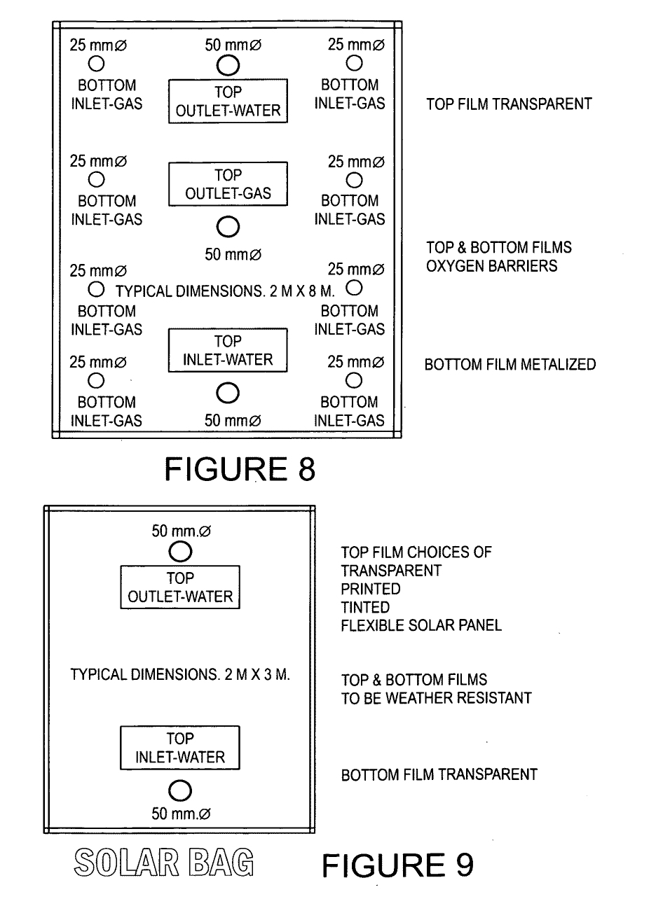

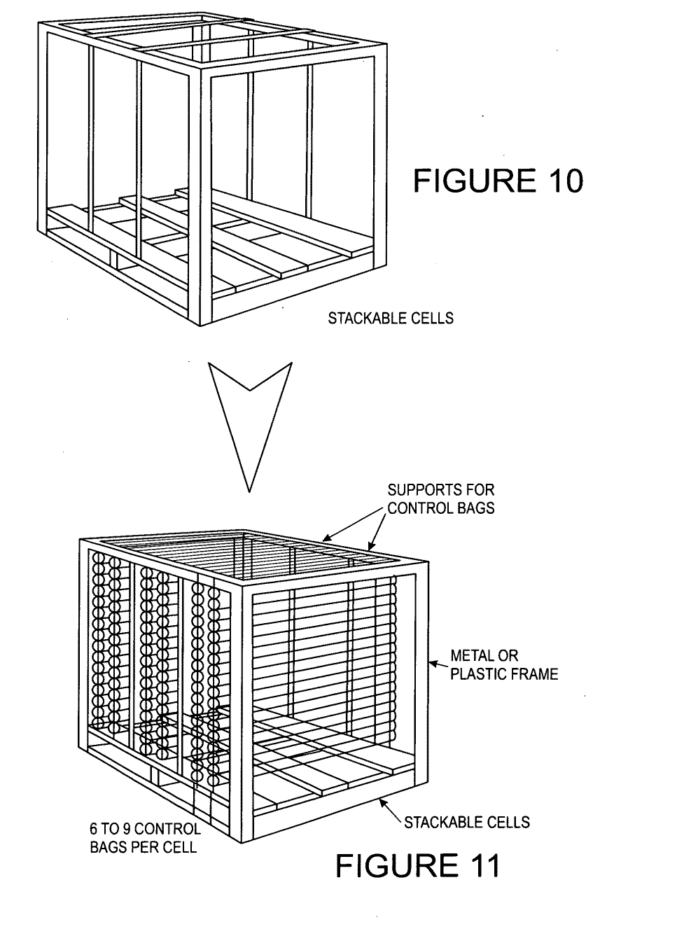

In the system five different forms of bags are used including two for the algae control stages and 3 for the algae farming stages. In the algae control stages such as shown in FIGS. 10 to 13 the cultivation bag and a control bag is required. In the algae farming stages such as shown in FIGS. 1 to 9 a phyto bag, and the solar bag and a temperature control bag are required for one embodiment of the invention.

The function of the algae control stages is to supply enough algae starter culture to allow algae farming production as soon as possible after the beginning of the anticipated start of optimum growing season. This growing season will be dependent on climatic conditions at the required location and dependent on the type of algae being farmed. For the algae control includes preparation and supply of the nutrients for the algae to be a farmed.

This algae control stage therefore is a maximising support for a collection of algae farmers and also a central research, development and processing of the algae.

The components required at the algae control stage include:

Cultivation bags

Control bags

Control cells

Batching, mixing and packing equipment for nutrient supply to farm

Temperature control equipment

LED lights

Dosing equipment

Transfer air diaphragm pumps

Air blowers

air filters and scrubbing equipment

Laboratory

Referring to FIGS. 10 to 13 the function of the cultivation bags used in the algae control is to grow initial starter culture that will be transferred to the control bag. The cultivation bag will be further used for nutrient supply to farm. The control bags are used to grow sufficient starter culture that can be transferred to the algae farm.

The control bags are designed to hang within control cells as shown in FIGS. 10 and 11 which are themselves stackable to optimise available space in a control facility. The control cells include use of artificial light from LED lights mounted within the control cell as shown in FIG. 11 with specific light and heat outputs as required by the particular algae species being grown.

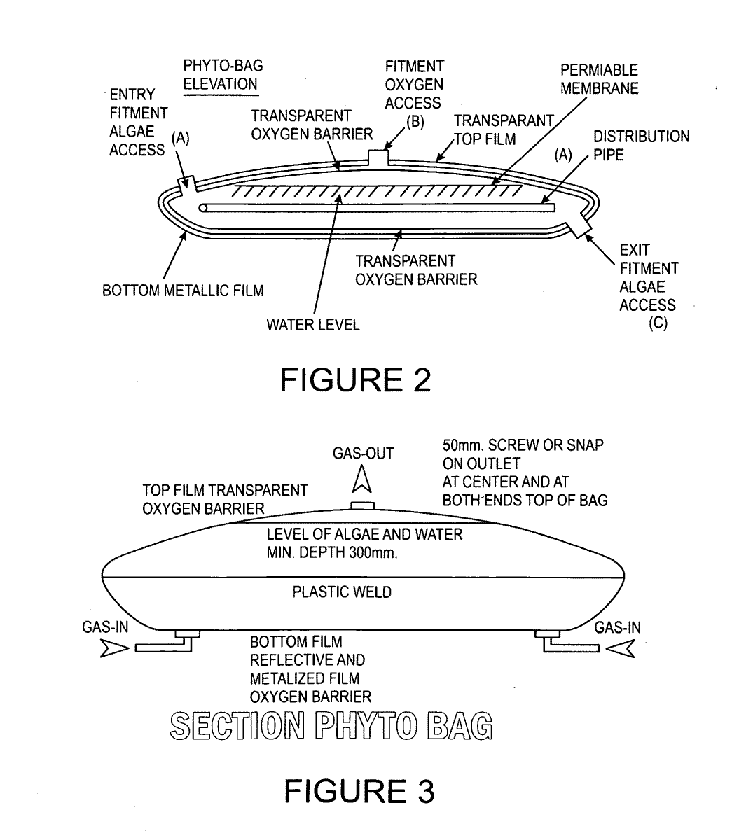

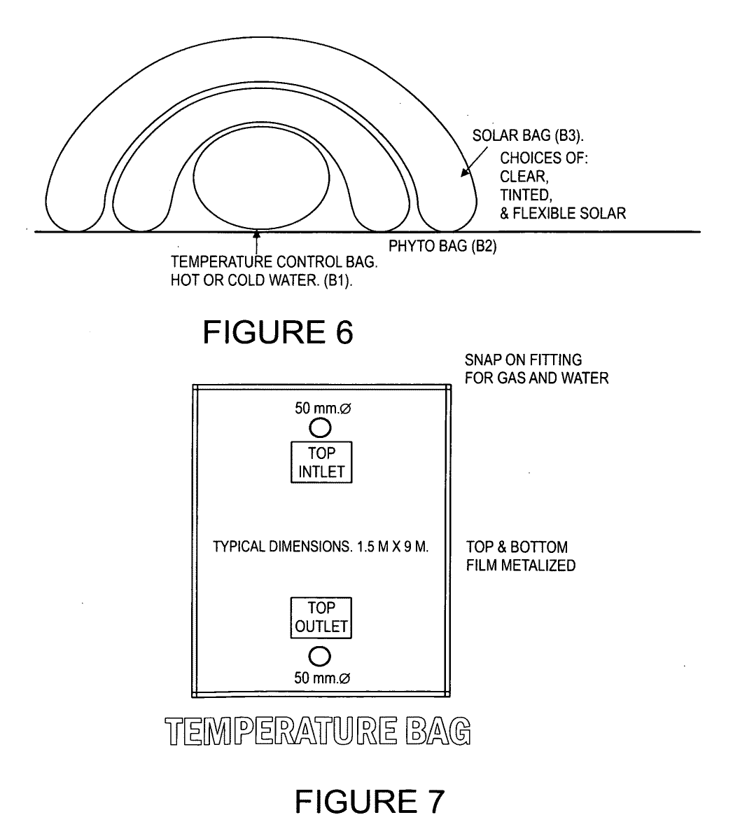

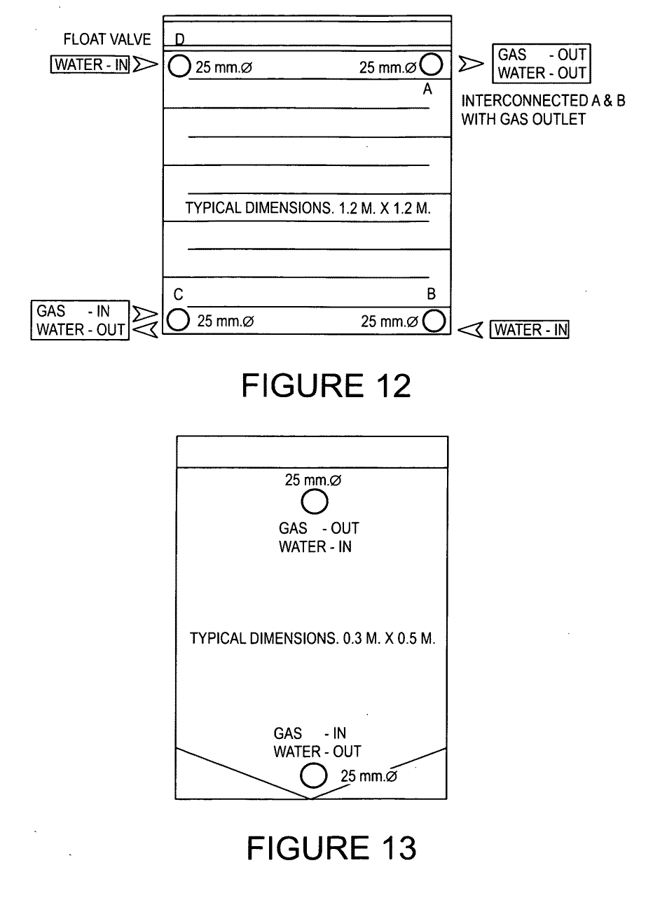

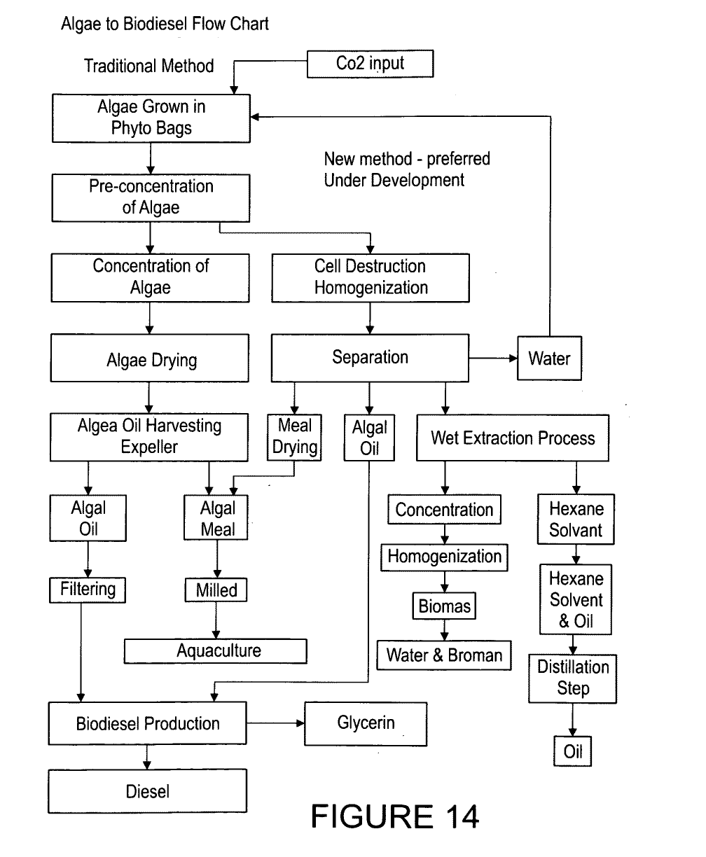

Both the cultivation bag and the control bag as shown in FIGS. 12 and 13 has a capacity to grow monocultures of different algae species within the same control facility. The control cell of FIG. 11 in the algae control stage is divided into support racks to hang control bags in cultivation bags. The control cells are also stackable to maximise in limited space available in the control facility to obtain sufficient quantity of stock of the starter culture required for multiple algae farms. The control cell also provides support for installation of LED lights. The control cells are readily transportable and as such provide a modular function and ready rearrangement within a limited space. The next stage of the process is the algae farming stage as shown in FIG. 1 and using the bags of FIGS. 2 to 9. Fundamentally the algal farming system enhances growth of algae for harvesting to form biofuels by using three forms of bags as shown particularly in FIG. 6. It includes a phyto bag having a large footprint relative to its height a top with a substantially translucent surface material. The solar bag lays over the phytpo bag and provides a sunlight controlling means on or above the top surface of the phyto bag. Also a temperature bag acts as a heat aiding means below the bottom surface of the phyto bag. Together the combination of three bags provides an effective the control of the sunlight controlling means and the heat aiding means ensuring heat control within the phyto bag to ensure heat in a predefined range to enable algae growth.

The solar bags used provide insulation to the phyto bag to minimise the amount of heat lost to atmosphere. Solar bags also provide a light filter to limit excessive light penetrating to the phyto bag. Tinting of the upper layer of the solar bags will vary according to conditions at farm locations. Solar bags also have the feature of having flexible solar panel laminated to a top layer of the bag as a source of energy and component to a connected hybrid renewable energy system that can be used to control the energy use of the algal farm system.

Also a smaller solar bag will have the function of being used as a drying vessel as shown in FIGS. 16 and 17 with dry air pumped in and wet air removed and dried with use of a condenser. Electrical heat pads will be used underneath the bags as a heat source supplementing the solar heat from sun. Operating temperature in the bag can be 60 degrees Celsius.

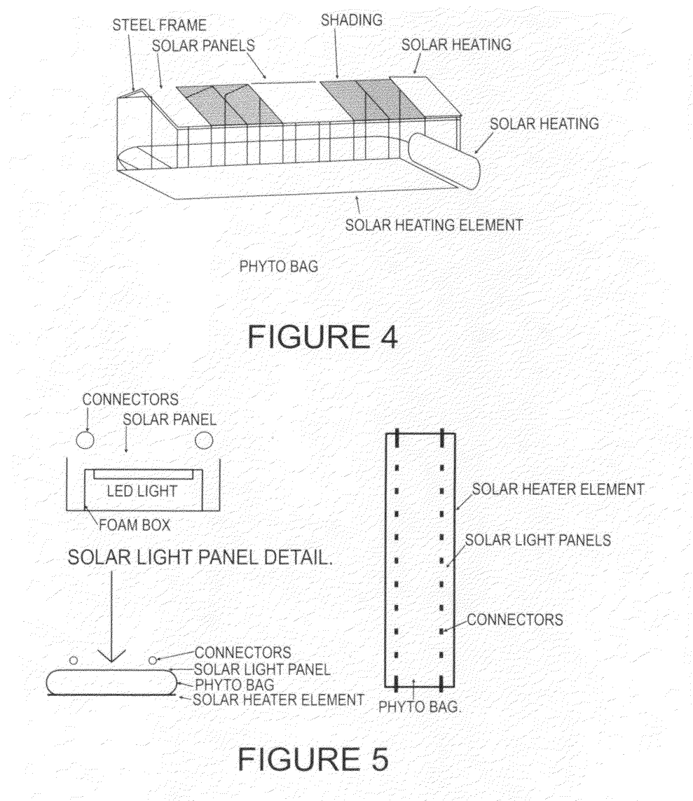

The phyto bag of FIG. 4, 5 or 6 are used to provide a protected environment for optimal growing conditions for monoculture algae.

Connections to the three types of bags of FIG. 6 are shown in FIGS. 7, 8 and 9. Phyto bags are fitted with inlet and outlet liquid transfer ports with snap on fittings and are used for delivery of gases containing CO₂ and providing physical agitation for the algae to remain in suspension within the fluid in the phyto bag. The bag is also fitted with ports for outputting oxygen rich gas to be collected for combustion or other uses. The phyto bags have a metallised bottom layer to reflect light back into the algae in liquid suspension in the bag.

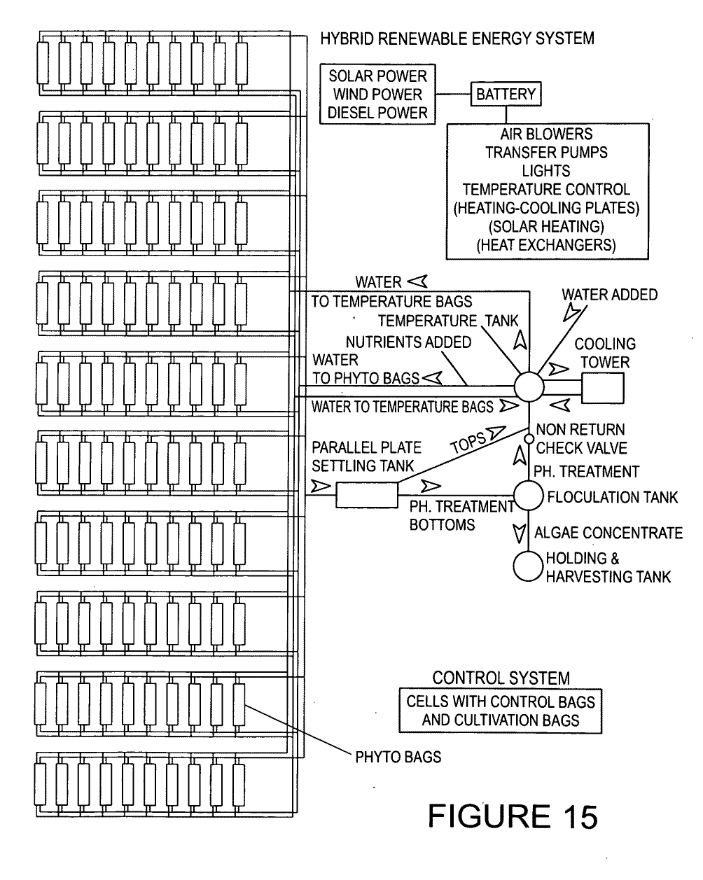

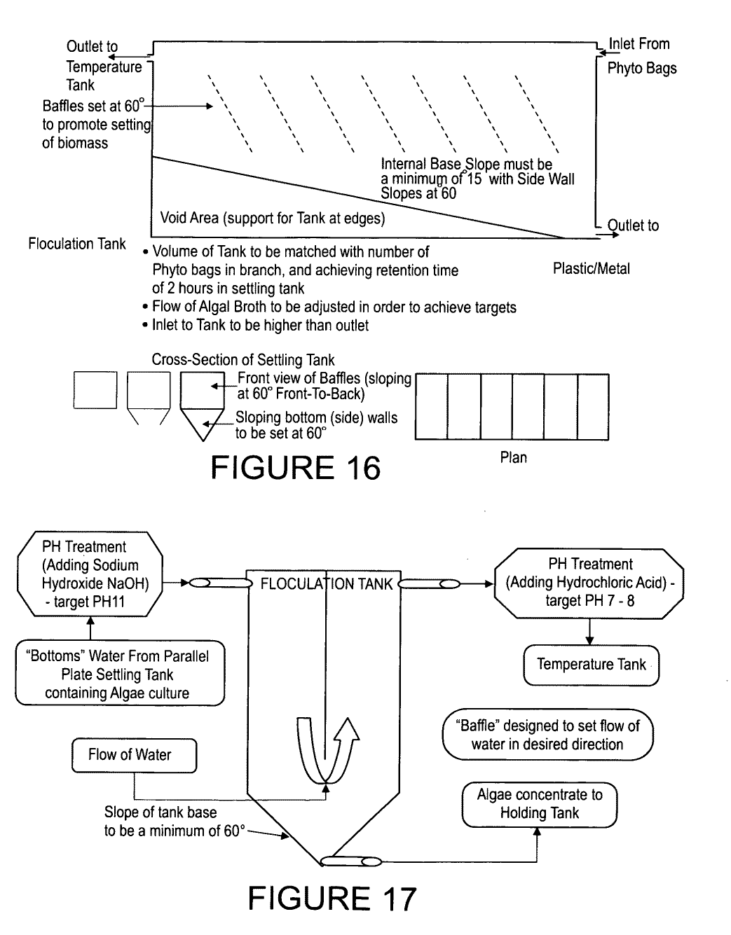

Referring to FIG. 15 the number of branches of an algae farm bag system is equal to the number of days required for algae biomass to double immediately prior to harvest which relates to the optimal algae density or 5 percent of liquid. Harvesting can potentially happen every day in a growing season given that all proper procedures are followed and algal biomass is maintained at 50 percent of optimum in the branches.

The nutrient supply system includes the algae monoculture being supplied from control location in control cells containing 10 control bags for each cell. Nutrients are prepared in control location and supplied to the farm location in cultivation bags. Each cultivation bag supplies a branch of phyto bags with preferably 10 phyto bags per branch. Nutrients to be dosed into solution after the temperature tank as phyto bags are refilled in series during harvesting process. Dosing mechanism will be a positive displacement pump dosing nutrient controls at required time intervals determined by a timer device.

The algae concentration system is required so the algae is harvested from phyto bags having concentration at least 5 percent biomass in solution. The objective of harvesting is removed 50% of biomass from phyto bags at time of harvesting. Remaining 2.5 percent biomass in solution, when returned from harvesting process would have nutrient added prior to being returned to phyto bags for next harvest cycle.

The harvesting cycle procedure is on a rotational basis to enable batching of branches. Therefore the number of days required for algae to regenerate to 5 percent biomass would determine the number of branches required. Harvesting requires the branch to be isolated and contents of phyto bags from that branch to be emptied in series directly into parallel plate settling tank through a positive displacement pump. The settling tank may be aboveground or below water level of phyto bags.

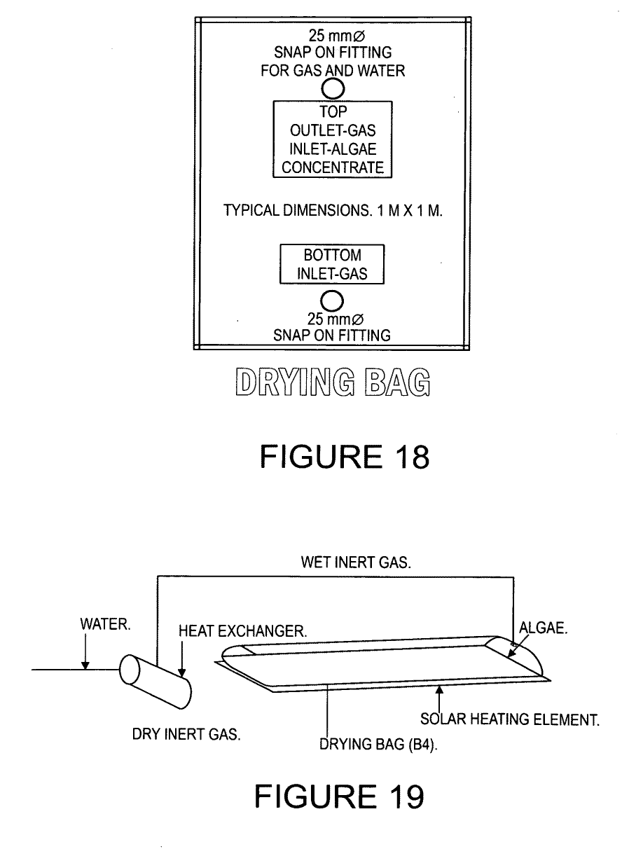

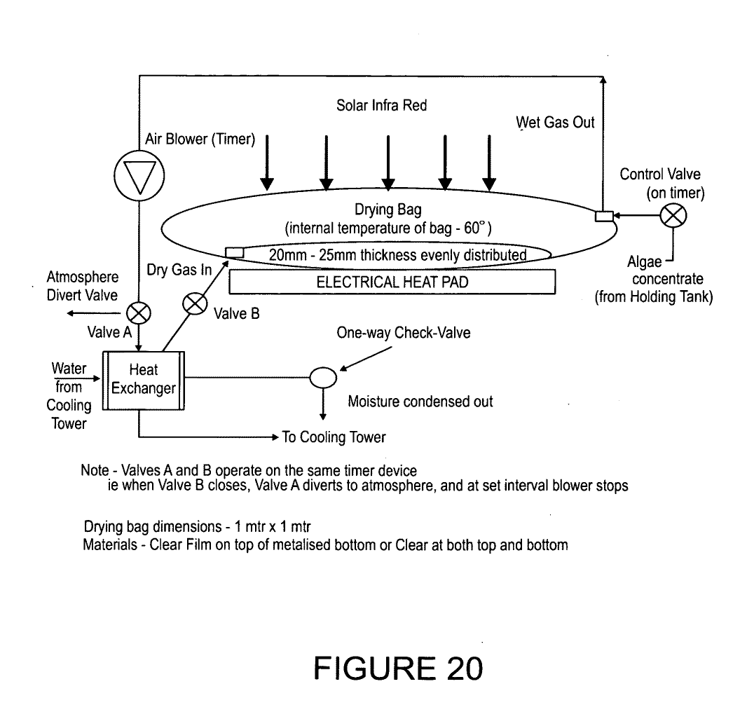

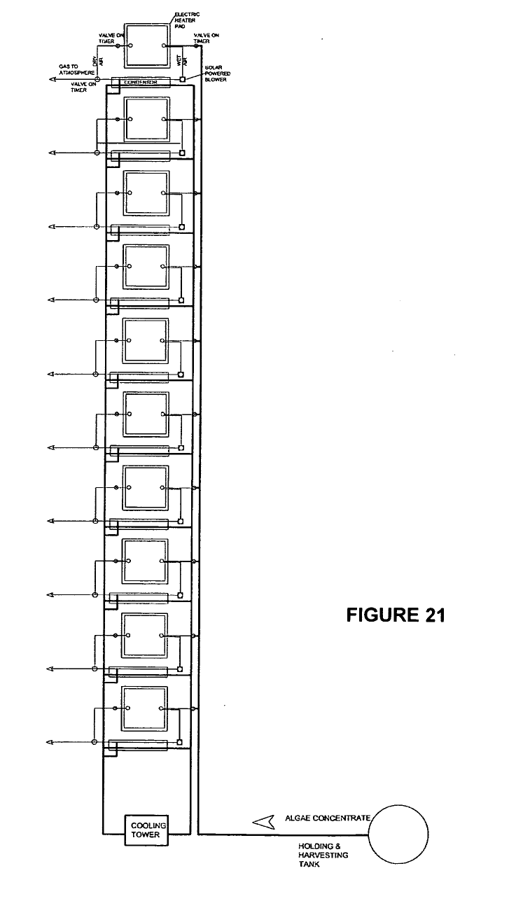

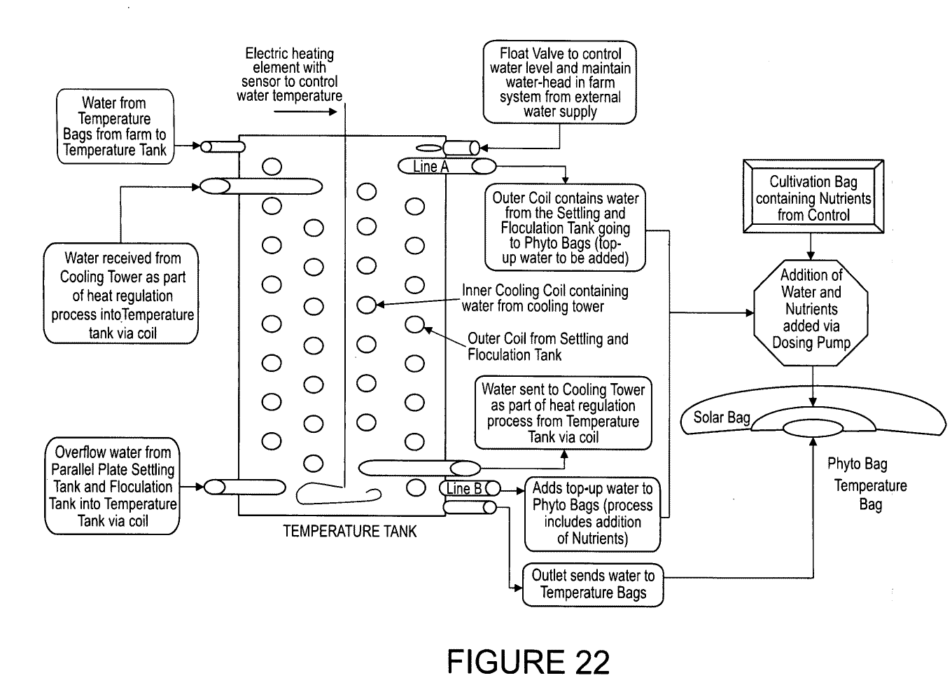

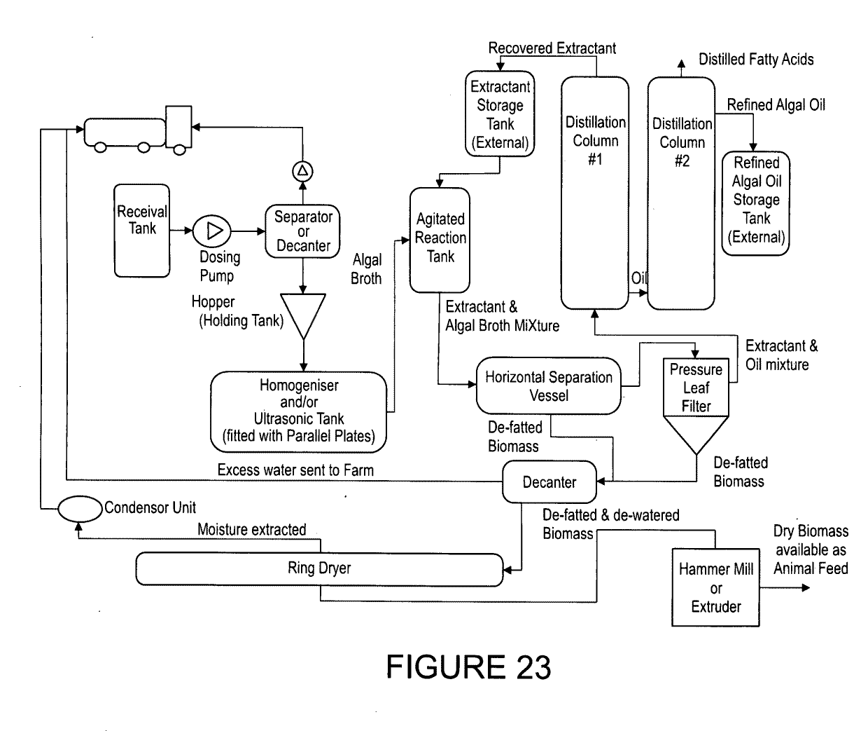

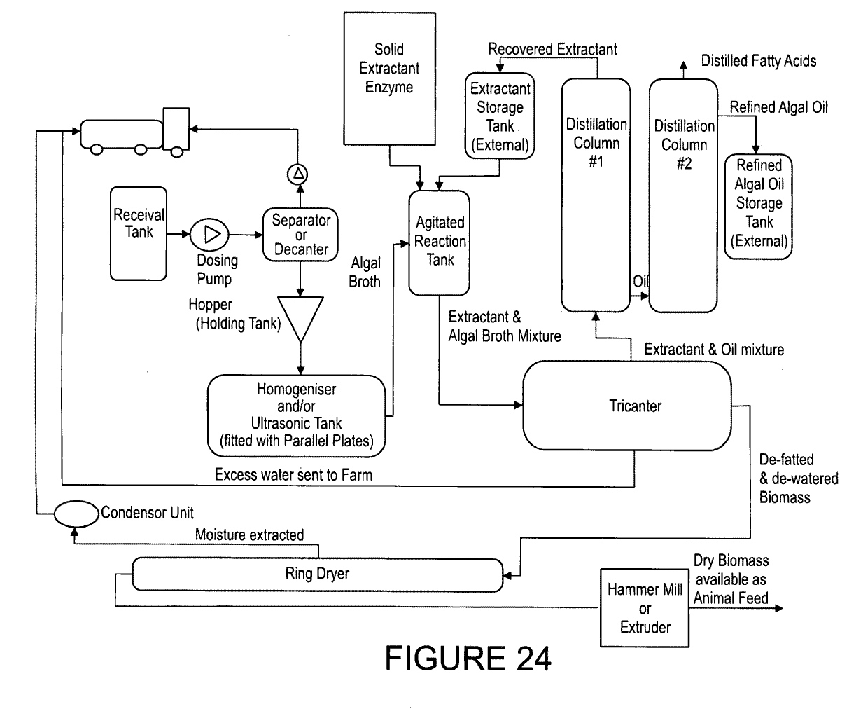

Referring to FIG. 18, algal overflow containing low concentration algal biomass (“tops”) once it leaves the parallel plate settling tank is diverted to inlet of temperature tank. Algal overflow containing high concentration algal biomass (“bottoms”) on leaving the parallel plate settling tank point is first treated with sodium hydroxide dosed in line using a dosing pump system controlled on a timer to achieve a pH of 11.

The bottoms enter a flocculation tank when the biomass and water are separated. The water flows through another dosing system also using a dosing pump controlled on a timer to bring it to a pH of 8 by adding Hydrochloric acid. The resultant balanced water goes through a non return check valve and pipes feed the line of tops to the temperature tank. The bottoms (or algae concentrate from the flocculation tank) are stored in the holding tank to be collected and taken to the extraction plant.

The hybrid renewable energy system includes the elements of FIG. 15 that assists the powering of the algal farming system includes:

SOLAR POWER

WIND POWER

DIESEL POWER

MAINS POWER

BATTERY FOR POWER STORAGE

The solar power systems will include flexible solar panels which are laminated to the solar bags. It also includes solar lighting panel placed on to phyto bags and secured via interconnecting delivery pipes providing direct heating to and from the algal harvesting system. Solar heating is locatable to support the shading system. The circulation pumps and the air blower pumps can also be solar powered. All of the above are designed and engineered according to energy requirements for each locality within the overall energy plan of the farm

Wind power can be a modular design in order to provide energy that would be stored in batteries and utilised for illumination especially at night or times of low sunlight. All of the above are designed and engineered according to energy requirements for each locality and to fit within the overall energy plan of the farm.

Diesel power is only to be used as a backup to solar and wind power generation. Any use of diesel will be preferably bio diesel and where possible use of glycerol as the fuel.

Mains power will be a last resort.

Clearly batteries are required to manage and store peak power production from all sources and will be designed in accordance with the overall energy plan for the farm.

The temperature control system includes the elements of:
Temperature Bag
Temperature Tank
Heating and Cooling Electrical Pads
Passive Control by Prevention by Solar Bags, Solar Panels, Shading System, Solar Water Heating and external Waste Heat Sources.
Cooling Tower

The temperature control bag is located preferably under the phyto bag only when required and dependent on local conditions. The solar bag always is utilised although tinting level will vary dependent on local conditions. The temperature bag will be linked fluidly to the temperature tank and will be controlled by heating element and cooling coil within the tank. A cooling tower with the source of cool water assists in the temperature control process by the cooling coil located in the temperature tank.

The optimum temperature range for algal farming is between 20 degrees Celsius and 30 degrees Celsius with critical points at 5 degrees Celsius and 38 degrees Celsius. In order to avoid critical points heating and cooling pads can be used as an alternative to temperature bags. These are electrically operated and have the capacity to heat on one side while cooling on the other. By reversing polarity heating and cooling will occur in reverse.

The solar panels placed on the phyto bags can include LED lights positioned directly underneath and therefore supplement the shading of the algae in extreme heat or via infrared light provide the required added energy for illumination by lights. The shading systems for the design of positioned to achieve dark spots which can be adjusted by orientation of the bag relative location of the shading system to the bag or variation of the material. The LED lights will have power illumination of 550 lux or 260 lumens with an operating temperature of 20 to 40 degrees Celsius and the power usage of 5 to 7 watts at 12 Volts.

Solar water heating is used in extreme cold climates which has a capacity up to 60 degrees Celsius. The waste heat can be recovered by heat exchangers or heating coil connected to the temperature tank.

The gas control system includes the controlling of:

DOSING OF CO₂

COLLECTION OF OXYGEN

AIR-BLOWERS

The dosing of CO₂ occurs by adding into the gas line which is connected to the phyto bag via a series of snap fittings at approximately 1.5 m along the length of the bag.

The collection of oxygen is achieved by the phyto bag having oxygen barriers and the top and bottom surface along with a central top discharge. At this point a collection system can be attached for the oxygen rich gas to be extracted, compressed and stored in an oxygen receiving tank for use as a combustion source or otherwise as required.

An air blower is allocated to each branch and when required with air filter to inlet of blower. The air blower enables circulation and agitation.

The fluid transfer system comprises:

Pumps (Solar AND Air Powered)

Dedicated Pipes

Flexible AND Solid Pipes

Positioning of Tanks to Maximize Static Head and Assisting Fluid Movement

Control Valves

A solar powered positive displacement pump is allocated to each branch. Pumps are allocated as required and dedicated to specific tasks and controlled by electronic switchboard. The task can include use in settling, flocculation or temperature tanks as well as a holding tank and cooling tower.

Dosing pumps are used for controlling liquid and gases and are operatively controlled via timers.

Dedicated pipes are utilised in order to eliminate cross contamination in the event of viral outbreak in any one branch or individual phyto bag. Flexible and solid pipes are used according to pressure and suction requirements pre and post pumps. Preferably flexible hosing and snap on fittings will be used.

Positioning of tanks will be such that the maximum static head is achieved to minimise pumping requirements. This is achieved by maintaining the water level in the temperature tank via float valve and keeping phyto bags and constant levels as well as having the settling tank below the water level of the phyto bags.

Control valves are used to allow the delivery of algae from phyto bags in series and operated by timers. Control valves also open to allow return from harvesting system back to the phyto bags and are controlled by the same timers.

The third part of the system as shown in FIGS. 16 and 17 is the full fat algae drying system which uses solar drying bags on a single use basis. The bags are designed to remove moisture from algal concentrate sourced from holding tank located on the farm. The quantity of concentrate metered into a drying bag is controlled and with timer is coupled to gas outlet of the drying bag. The concentrate is delivered to a drying bag from a branch and pumped into that bag by positive displacement pump. Moist gas is extracted by blower system operating intermittently on a timer basis is connected through a heat exchanger. The heat exchanger is cooled from water source from cooling tower such that moisture is condensed by the head exchanger and piped into return waterline via a non return checks valve to the cooling tower. Drying gases flow back into the solar drying bag to complete the cycle.

Heat is added to system via solar infrared rays sourced from the sun and electrical pads placed underneath drying bags into powered by the hybrid energy system.

The concentrate remains in the bag as a full fat biomass with all air expelled from drying bags by shutting inlet valve and diverting air to atmosphere, again operated by timer system. Dry bags are then collected and flat packed will transport. Expected maximum dry weight of biomass for bag is 15 kg.

### EXAMPLE 1

In one particular example of the invention the algae used is of the species Nannochloropsis Oculata. This includes features of
a nonmotile greenish coloured cell with flagella
a small cell, 4-6 μm in diameter.
Cells tend to float in culture and stay in suspension without aeration.

The required growing conditions are:
Temperature 20-30° C.
Light 2500-6000 Lux
pH 7.5-8.5
Salinity 10-36 ppt

The nutrient requirements are:
NaNO₃-150 mg/l
NaHPO₄-8.69 mg/l
Ferric EDTA-10 mg/l
MnCl₂-0.22 mg/l
CoCl₂-0.11 mg/l
CuSO₄, 5H₂O-0.0196 mg/l
ZnSO₄, 7H₂O-0.044 mg/l
Na₂SiO₃, 2H₂O-60 mg/l
B₁₂-1.0 μg/l
Biotin-1.0 μg/l
Thiamine HCI-0.2 mg/l

When CO₂ is used, a common practice is to inject intermittently using a timer and solenoid valve to maintain pH between 7.5 and 8.5. Typically require 1-1.7 kg of CO₂ to produce 1 kg of algal bio-mass.

Oil content of Nannochloropsis is 31-68 (% dry weight).

The final selection of algae used at each location will generally be influenced by the “naturally occurring” variety in the vicinity, taking into account factors such as oil yields and other desired properties.

Algae Recovery and Algal Oil Extraction Process

A) Algae Recovery

The Algae is grown in a Farm consisting of multiple bags of Algae in Water. This Algae water solution is pumped from selected bags (harvested) when fully grown and separated from the water in a Parallel Plate Separator or similar gravity settling vessel of sufficient size for the farm in question. The excess water and overflow of Algae is returned to the farm via temperature tank.

The concentrate from this is pumped to a second settling vessel or flocculation tank and on the way to the vessel the pH is adjusted to promote further settling and concentration to minimize water transportation. The excess water is then dosed to neutralize the pH and returned to the farm via temperature tank.

Alternatively the concentration steps could be achieved through either high speed decanter centrifuges or disc centrifuges.

When sufficient concentrate is gathered it is transported to the processing plant either via trucks or pipelines depending on the distance involved.

B) Algal Oil Extraction (Wet Extraction Process)

The concentrate is unloaded into a storage or receivals tank. It is then either homogenized or treated in an ultrasonic treatment vessel at pressures in excess of 5000 psi to aid in the opening of the cell walls and free the oil within. This is then pumped to an extraction vessel.

The extractant is then added to the algal concentrate and agitated for a period to allow the reaction to take place. The oil/extractant mixture is then separated from the remaining biomass and water either using gravity settling (separation vessel) or centrifugation. The oil/extractant mixture is then pumped to a first distillation column where the extractant is recovered and the oil is then sent to the second distillation column for fatty acid separation. Desirable triglycerides sent to biodiesel plant for transesterification (i.e. biodiesel production). The extractant is reclaimed in first distribution column and returned to extraction vessel.

The underflow of the separation vessel or the discharged biomass from the separator is then sent to a drying plant to dry the biomass for use as an animal feed additive for instance (if a gravity settling vessel was used to recover the oil/extractant mix then it is likely necessary to have a second gravity separation vessel in order to lower the amount of water going to the dryer).

In either case mechanical pre-dewatering using centrifuges or filters prior to drying normally has advantages.

Other processes that can be adopted or applied to deal with de-fatted biomass:
Pasteurisation for liquid animal feed
Anaerobic digestion for methane gas production (“bio-gas”).

Detailed Description of Process

A-Pre Treatment
1. Receivals tank 30 Metric ton capacity—Holding tank to receive Concentrated Algae Bio Mass (“CAB”) from road tanker
2. CAB pumped by variable speed positive displacement pump fitted with dosing device for PH control.
3. Centrifuge (Decanter or Separator) to remove excess water (back to farms)
4. Buffer tank and centrifical pump
5. Homogenizer and/or ultrasonic tank fitted with parallel plates where pressures in excess of 5000 psi are achieved in solution. The resulting product is now referred to as “Algal Broth”.

B-Extraction (Process & All Motors to be Explosion Proof)
6. Algal Broth is pumped by positive displacement air pump into agitated reactor tank with capacity to provide vacuum.
7. Extractant dosed into Algal Broth from Extractant storage tank and mixed in reactor. Mixture then pumped to either horizontal separation vessel or tricanter (centrifuge).
8. Horizontal Separation Vessel (fitted with top and bottom take-off points):
   Tops (extractant and algal oil) pumped by variable speed positive displacement pump to pressure leaf filter
   Tops to first distillation column, where extractant is recovered at the top of the distillation column and sent to storage tank and algal oil is recovered at the bottom of the distillation column.
   Algal oils then sent to second distillation column for fatty acid separation
   Refined algal oil sent to storage
   Bottoms from horizontal separation vessel can be:
      a. pumped to decanter to remove excess water (this water then sent back to farms)
         i. The resulting wet cake sent to ring dryer where no more than 6%-8% moisture content remains
         ii. Dry cake is then hammer milled or extruded
      b. treated by pasteurization (ie bottoms heated to 130° C. and cooled to 30° C. and packed in hygienic sealed bags)
      c. digested in anaerobic conditions to produce bio-gas (methane)
9. Tricanter (explosion proof)
   Tops (solvent and algal oil) to distillation
   Steps as per point 8 above
   remove excess water (this water is sent back to farms)
   Bottoms to be treated as per steps at point 8 above

It should be understood that the above description is of a preferred embodiment and included as illustration only. It is not limiting of the invention. Clearly variations of the method and apparatus of algal oil production would be understood by a person skilled in the art without any inventiveness and such variations are included within the scope of this invention as defined in the following claims.

1. A method of algal oil production including the steps of:
a. control growing to provide intensive growth to supply starting means for algae farming;
b. farming algae from using primarily sunlight
c. processing algae from
wherein at least one of the steps includes use of a bag able to be interconnected to gas or liquid flows of at least one of water, CO₂, oxygen or air, and wherein in a plurality of the steps of control growing, farming and processing, a specialised algae growing bag is used to enable ready batching, transport and interconnection into modular systems, wherein the specialised algae growing bag is a control bag having a serpentine pathway from lower inlet to upper outlet to promote flow and agitation of algae in a bag in a hanging position wherein CO2 and nutrients are more effectively infused to the algae solution.

2. A method of algal oil production including the steps of:
a. control growing to provide intensive growth to supply starting means for algae farming;
b. farming algae from using primarily sunlight
c. processing algae from
wherein at least one of the steps includes use of a bag able to be interconnected to gas or liquid flows of at least one of water, CO₂, oxygen or air, and wherein the step of processing includes substantially full fat production with algae retaining lipid content being processed by a drying process, and wherein the bag is a drying bag used in the drying process and allowing for ready transport of final product in a flat pack of multiple bags.

3. A method of algal oil production according to claim 1 wherein the step of processing includes substantially full fat production with algae retaining lipid content being processed by a drying process, and wherein the bag is a drying bag used in the drying process and allowing for ready transport of final product in a flat pack of multiple bags.

4. A method of algal oil production according to claim 1 wherein the step of processing includes a defatting production of algae with lipid content removed and being processed by a wet process including:
a. preconcentration of grown algae with a suspension moisture content of at least 50% to form a flowable liquid
b. physical breaking of the algal cells to release the lipid content such as by homogenising of the preconcentrated algae in liquid phase by high pressure in excess of 5000 psi and
c. chemical breaking of the algal cells for releasing lipid such as by adding solvent, enzyme protease and/or similar enzyme
d. adding extractant for removing the released lipids
wherein the physical and chemical breaking of the algal steps improves the effectiveness of lipid removal.

5. A method of algal oil production according to claim 2 wherein the step of processing includes a defatting production of algae with lipid content removed and being processed by a wet process including:
a. Preconcentration of grown algae with a suspension moisture content of at least 50% to form a flowable liquid
b. Physical breaking of the algal cells to release the lipid content such as by homogenising of the preconcentrated algae in liquid phase by high pressure in excess of 5000 psi and
c. Chemical breaking of the algal cells for releasing lipid such as by adding solvent, enzyme protease and/or similar enzyme
d. Adding extractant for removing the released lipids
wherein the physical and chemical breaking of the algal steps improves the effectiveness of lipid removal

6. A method of algal oil production according to claim 5 wherein wet process further includes steps of:
a. physical separation by centrifuge or horizontal continuous settling to separate out extractant and oil mixture from biomass and solid extractant, which is for later drying processing;
b. a first distillation process to remove the extractant from the extractant and oil mixture
wherein the extractant is recovered and can be recycled to system.

7. A method of algal oil production according to claim 6 wherein wet process further includes steps of:
a. a second distillation process to remove mono, bi and tri glycerides
b. outputting the refined algal oil for further processing
wherein the second distillation provides a refined algal oil that is suitable for biodiesel production and distilled fatty acids that are suitable for food production.

8. A phyto bag for enhancing growth of algae for harvesting to form biofuels including:
i. a bag made from a substantially flexible sheet material enabling ready construction of large quantities;
ii. the bag including a clear transparent top film to allow the passage of light to the algae in the bag; and
iii. a metallic reflective bottom film to reflect light back to the algae in the bag
wherein the bag has a large footprint relative to its height and the clear transparent top film and the metallic reflective bottom film improve sunlight and heat transfer to an algae material in the bag to enhance growth.

9. A phyto bag of claim 8 further including a solar bag having enclosed air cavity for providing insulation and having peripheral weights to allow ready location over the phyto bag
a. wherein the solar bag includes upper and lower translucent surfaces for allowing ready transfer of sunlight to the phyto bag; and
b. light filtering means.

10. The phyto bag in accordance with claim 8 including:
a. a clear transparent top film to allow the passage of light to the algae in the bag
b. a metallic and reflective bottom film to reflect light back to the algae in the bag
c. multiple points of attachment to access contents which can be the form of liquids or gases
d. and when the top and bottom films have a medium to high oxygen barrier to capture oxygen produced by the algae
e. multiple fluid delivery means consisting of pipes and chambers positioned within the bag to maximise agitation
f. a minimum footprint area of 1 sq m per phyto bag

11. The phyto bag in accordance with claim 9 including:
a. a clear transparent top film to allow the passage of light to the algae in the bag
b. a metallic and reflective bottom film to reflect light back to the algae in the bag
c. multiple points of attachment to access contents which can be the form of liquids or gases
d. and when the top and bottom films have a medium to high oxygen barrier to capture oxygen produced by the algae
e. multiple fluid delivery means consisting of pipes and chambers positioned within the bag to maximise agitation
f. a minimum footprint area of 1 sq m per phyto bag.

12. A method of algal oil production according to claim 1 wherein the phyto bag modular system consists of the plurality of bags that are interconnectable and in addition comprise:
a. aboveground tank fitted with heating and cooling options located an elevated position to achieve maximum static head
b. transfer pumps to effect fluid movements
c. bags placed on flat and sloping ground over pipe heat exchangers
d. below ground tank which would be the receival or harvesting tank

13. A method of algal oil production according to claim 1 including the steps of:
a. providing one or more phyto bags having ratio of footprint to the height of the order of greater than 30:1;
b. providing a heat control system for substantially controlling the temperature between 20 and 25° C.
c. providing a sunlight enhancement to the contents by the bag being constructed of material including translucent and reflective materials;
d. providing input of CO₂ at the required rate for growth of algae;
e. providing flow of saltwater with salinity substantially similar to seawater
wherein upon inclusion of algal material from the family of nanochloropsis an improved algae growth occurs.

14. A method of algal oil production according to claim 8 including the steps of:
a. providing one or more phyto bags having ratio of footprint to the height of the order of greater than 30:1;
b. providing a heat control system for substantially controlling the temperature between 20 and 25° C.
c. providing a sunlight enhancement to the contents by the bag being constructed of material including translucent and reflective materials;
d. providing input of CO₂ at the required rate for growth of algae;
e. providing flow of saltwater with salinity substantially similar to seawater
wherein upon inclusion of algal material from the family of nanochloropsis an improved algae growth occurs.

15. A method of algal oil production according to claim 1 including the steps of:
a. preconcentration of grown algae with a suspension moisture content of at least 50% to form a flowable liquid
b. physical breaking of the algal cells to release the lipid content such as by homogenising of the preconcentrated algae in liquid phase by high pressure in excess of 5000 psi and
c. chemical breaking of the algal cells for releasing lipid such as by adding solvent, enzyme protease and/or similar enzyme
d. adding extractant for removing the released lipids
wherein the physical and chemical breaking of the algal steps improves the effectiveness of lipid removal.

16. A method of algal oil production of claim 8 including the further steps of:
a. physical separation by centrifuge or horizontal continuous settling to separate out extractant and oil mixture from biomass and solid extractant, which is for later drying processing;
b. a first distillation process to remove the extractant from the extractant and oil mixture
wherein the extractant is recovered and can be recycled to system.

17. A method of algal oil production of claim 1 including the further steps of:
a. physical separation by centrifuge or horizontal continuous settling to separate out extractant and oil mixture from biomass and solid extractant, which is for later drying processing;
b. a first distillation process to remove the extractant from the extractant and oil mixture
wherein the extractant is recovered and can be recycled to system.

18. A method of algal oil production of claim 1 including the further steps of
a. a second distillation process to remove mono , bi and tri glycerides
b. outputting the refined algal oil for further processing
wherein the second distillation provides a refined algal oil that is suitable for biodiesel production and distilled fatty acids that are suitable for food production.
